(19) United States
(12) Patent Application Publication
(10) Pub. No.: US 2007/0249888 A1
(43) Pub. Date: Oct. 25, 2007

(54) Blood Pump-Oxygenator System

(76) Inventors: Zhongjun Wu, Woodstock, MD (US); James F. Antaki, Allison Park, PA (US); Bartley P. Griffith, Gibson Island, MD (US)

Correspondence Address: BARNES & THORNBURG LLP, P.O. BOX 2786, CHICAGO, IL, 60690-2786, US

(21) Appl. No.: 11/575,119

(22) PCT Filed: Sep. 13, 2005
(86) PCT No.: PCT/US05/32675

§ 371 Date: May 3, 2007

Related U.S. Application Data
(60) Provisional application No. 60/609,411, filed on Sep. 13, 2004.

(51) Int. Cl. A61M 37/00

(52) U.S. Cl. 600/16

(57) ABSTRACT

A blood pump-oxygenator system including a housing, an impeller, a fiber bed, and a bypass channel that provides a path for blood to be recirculated through the fiber bed; a system comprising a housing, a means for drawing blood into the housing, a means for removing carbon dioxide from the blood, a means for adding oxygen to the blood, and a means for recirculating the blood back through the removing means and the adding means; and a method for oxygenating blood comprising drawing blood into a housing comprising a fiber bed, propelling blood principally in a radial direction through the fiber bed, adding oxygen to the blood as it moves through the fiber bed, and repeating the forcing and adding steps for at least a portion of the blood.

### CROSS-REFERENCE TO RELATED PATENT APPLICATIONS

This patent application claims the benefit of U.S. Provisional Patent Application No. 60/609,411, filed Sep. 13, 2004.

### FIELD OF THE INVENTION

This invention relates to a compact artificial pump-lung system, more specifically an integrated pump and oxygenator that can be implanted in the body or externally as a paracorporeal heart-lung to provide respiratory support for patients with lung diseases or used as a heart-lung machine for cardiopulmonary support during open-heart surgery.

### BACKGROUND OF THE INVENTION

Lung disease is the third largest cause of death in the United States, accounting for approximately 1 out every 7 adult deaths. In fact, an estimated 30 million Americans are now living with chronic lung disease. Adult respiratory distress syndrome (ARDS), in this regard, afflicts approximately 150,000 patients annually in the U.S., and despite advances in critical care, mortality remains between 40% and 50%.

Currently available therapies for patients with chronic respiratory failure include, for example, ventilation and extracorporeal membrane oxygenation (ECMO). Often, however, the tidal volumes, airway pressure, and oxygen fraction necessary to achieve sufficient gas exchange with these therapies can cause further damage to the lungs creating ventilator-induced lung injury, including barotrauma, volutrauma, and other iatrogenic injuries, further exacerbating acute respiratory insufficiency in many patients. Conventional oxygenator systems can also be associated with such problems as a general complexity of operation, thrombosis, blood trauma, infection, bleeding due to the need for high levels of anticoagulation, and limited mobility of the patient.

Efforts to develop more efficient and compact pump-lungs for use in both respiratory support and cardiopulmonary support have been forthcoming. In particular, for example, there have been attempts to integrate multiple components of cardiopulmonary systems into single structures, thereby, for example, eliminating or minimizing the need for the extension of lengthy, blood-filled tubes. These types of integrated pump-oxygenators have been described, for example, in U.S. Pat. Nos. 5,217,689, 5,266,265, 5,270,005, and 5,770,149 to Raible, U.S. Pat. No. 4,975,247 to Badolato et al., U.S. Pat. No. 5,429,486 to Schock et al., and U.S. Pat. No. 6,730,267 to Stringer et al. Drawbacks associated with these integrated pump-oxygenators include, however, non-uniform blood flow through fiber membranes and the existence of laminar boundary flow zones between blood cells and fiber membranes. The non-uniform blood flow across the fiber membranes, in this regard, results in hyper- and hypo-perfusion of the blood in flow paths. Hyper-perfusion is defined as exposure of oxygen-saturated blood to oxygenator fibers, which does not grant any additional benefit yet exposes blood unnecessarily to elevated shear stress and synthetic material contact. Hypo-perfusion is defined as the incomplete saturation of blood prior to discharge from the oxygenator. In order to ensure that all blood cells in a hypo-perfusion region are well-oxygenated, longer flow paths are needed, thus resulting in extended blood contact with the fiber membrane surfaces and requiring the fiber membranes to have a large surface area. Unfortunately, these are the major contributing factors to blood activation and, consequently, to thrombosis formation. When the blood is passively pumped to flow through fiber membranes, a relatively thick blood boundary layer is developed. The blood boundary layer increases the resistance to oxygen diffusion to blood cells that are not directly in contact with fiber membrane surface. Thus, gas transfer efficiency is significantly hindered by the existence of the boundary layer. Therefore, gas-exchange membrane surface areas of 2 to 4 m² are typically required to provide the needed gas exchange.

Efforts to decrease the boundary layer effect have been forthcoming. In particular, for example, some have sought to increase the shear rate and/or turbulence of the blood flow path by the introduction of secondary flows, for example, by directing blood to flow perpendicular (or at a substantial angle) to the fiber membranes. U.S. Pat. No. 4,639,353 to Takemura, for example, discloses the use of an arrangement of bundles of hollow fibers perpendicular to the direction of blood flow via a series of flow guide structures. Moreover, U.S. Pat. No. 5,263,924 to Mathewson describes an integrated centrifugal pump and membrane oxygenator comprising hollow fibers that are displaced circumferentially in a ring around an impeller of the centrifugal pump, and through which blood is pumped for oxygenation. Attempts have also been made to reduce the boundary layer effect by actively rotating hollow fibers membranes or by causing motion of fiber membranes in blood flow. This results in the relative motion of membrane surfaces to the blood cells, which can cause the pumping of blood and oxygenation of the blood to occur simultaneously and can disrupt the buildup of the boundary layers around the gas-exchange surface. Examples of oxygenators with active gas-exchange membranes include those described in U.S. Pat. No. 5,830,370 to Maloney et al., U.S. Pat. No. 6,723,284 to Reeder et al., U.S. Pat. No. 6,503,450 to Afzal et al., and in the paper by Makarewics et al. (ASAIO 42: M615-619, 1996).

Despite improvements in the performance and design of conventional oxygenator systems and devices, there remains a need for more compact and efficient blood pump-oxygenator systems and methods, in order to enhance the treatment of patients having lung disease and/or cardiovascular disease.

### BRIEF SUMMARY OF THE INVENTION

The present invention provides a blood-pump oxygenator system comprising a housing, an impeller disposed within the housing, a fiber bed disposed between a wall of the housing and the impeller, and a bypass channel that provides a path for blood to be recirculated through the fiber bed.

Moreover, the present invention provides a blood pump-oxygenator system comprising a housing, a means for drawing blood into the housing, a means for removing carbon dioxide from the blood, a means for adding oxygen to the blood, and a means for recirculating the blood back through the removing means and the adding means.

The present invention also provides a method for oxygenating blood, comprising drawing blood into a housing, forcing the blood to move radially outward through a fiber bed, adding oxygen to the blood as it moves through the fiber bed, and, for at least a portion of the blood, repeating the forcing and adding steps.

These and other aspects of the present invention will become apparent from the following description when taken in connection with the accompanying drawings which, for purposes of illustration only, show embodiments in accordance with the present invention.

### BRIEF DESCRIPTION OF THE DRAWINGS

FIG. 1 is a diagram depicting a blood pump-oxygenator system configured according to an embodiment of the invention.

FIG. 2 is a diagram depicting a blood pump-oxygenator system configured according to an alternative embodiment of the invention.

FIG. 3 is a diagram depicting a blood pump-oxygenator system configured according to an alternative embodiment of the invention.

FIG. 4 is a diagram depicting a blood pump-oxygenator system configured according to an alternative embodiment of the invention.

### DETAILED DESCRIPTION OF THE INVENTION

The present invention provides a system comprising a housing, an impeller disposed within the housing, and a fiber bed disposed between an inner wall of the housing on the impeller. A bypass channel is defined by a wall of the housing and an outer periphery of the fiber bed, wherein the bypass channel provides a path for blood to be recirculated through the fiber bed. Another blood pump-oxygenator system is also provided. The system comprises a housing, a means for drawing blood into the housing, a means for removing carbon dioxide from the blood, a means for adding oxygen to the blood, and a means for recirculating the blood back through the removing means and the adding means.

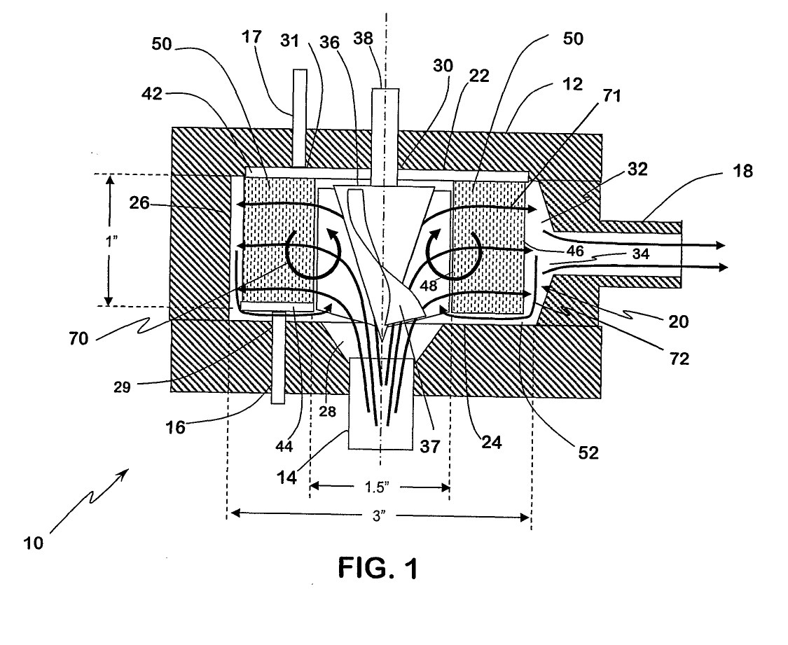

FIG. 1 illustrates a blood pump-oxygenator system in accordance with an embodiment of the present invention. In particular, the system 10 includes a generally cylindrical housing 12, which includes a blood inlet 14, an oxygen inlet 16, a carbon dioxide outlet 17 and a blood outlet 18. Although the blood inlet 14 is depicted in FIG. 1 as being oriented along the vertical axis of the housing 12, other orientations are possible. For example, in one embodiment, the blood inlet 14 has a low-profile configuration, in which it is oriented perpendicular to the vertical axis of the housing 12, such as described, for example, in U.S. Pat. App. Pub. No. 2003/0233144 A1, the contents of which are incorporated herein by reference in their entirety. The exact configuration of the low profile inlet, in this regard, can be computationally optimized.

Within the housing 12 is a main chamber 20 defined by inner surfaces or walls of the housing 12. These inner surfaces include a ceiling 22, a floor 24, and a sidewall 26. The floor 24 is generally circular, and has a frustoconical opening 28 in its center, through which the blood inlet 14 communicates. The floor 24 also has an off-center opening 29 through which the oxygen inlet 16 passes. The ceiling 22 is generally circular, is generally parallel to the floor 24, and has an opening 30 in its center for receiving the shaft of an impeller (described below). The ceiling 22 also has an off-center opening 31, through which the carbon dioxide outlet 17 is exhausted. The sidewall 26 is generally curved and extends around the housing 12. The sidewall 26 is disposed between, and is contiguous with the ceiling 22 and the floor 24. One portion of the sidewall 26 flairs out into a frustoconical portion 32 having an opening 34 that communicates with the blood outlet 18.

The pump-oxygenator system 10 also has an impeller 36 disposed approximately in the center of the main chamber 20 of the housing 12. The impeller 36 has a substantially conical hub at one end, which has an open nose. At its other end, the impeller 36 is supported by a shaft 38 that extends through the opening 30 of the ceiling 22. A lip seal made of elastomeric material engages the shaft 38 at the opening 30 to prevent blood from leaking from the housing 12. The impeller 36 can also comprise blades 37, which are situated on the outer surface of the impeller 36 and which can be curved in a radial and/or a circumferential direction, as illustrated in FIG. 1, and as discussed further below. The shaft 38 is coupled to an external motor assembly, which is not shown. The external motor assembly includes a motor and drive circuitry. A purge flow system can be used to provide lubrication fluid to the shaft 36, as well as the surface of the lip seal.

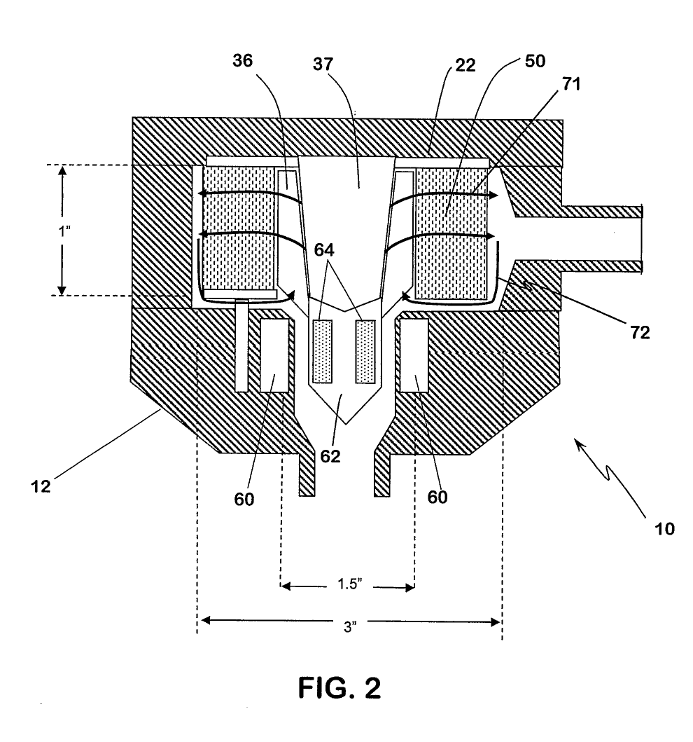

The pump-oxygenator system 10 also has a gas transfer, hollow fiber bed 50. The fiber bed 50 is generally annular-shaped and has an outer periphery 46 and an inner periphery 48. In one embodiment, the height of the fiber bed 50 is about 1 inch, the diameter of the inner periphery 48 is about 1.5 inches, and the diameter of the outer periphery is about 3 inches, as is illustrated in FIG. 1. The impeller 36 is disposed within the inner periphery 48 of the fiber bed 50, and is optimized for pumping blood uniformly through the fiber bed 50. During operation of the pump-oxygenator system 10, the impeller 36 rotates. The resulting circumferential motion of the blood (depicted by directional arrow 70) increases the velocity of the flow, thereby increasing the gas exchange efficiency of the fiber bed 50. The passage of the blades 37 of the impeller 36 relative to the fiber bed 50 also generates a secondary flow to disrupt the formation of a boundary layer. In determining the specific dimensions and configuration of the pump-oxygenator system 10, the velocity and the incident angle of the blood flow to the fiber bed 50 are determined by optimizing the required exposure time of the blood cells to the fiber bed 50 and by the thickness of the fiber bed 50. Preferably, the blood cells are exposed to the fiber bed 50 just long enough to be completely oxygenated. The thickness of the fiber bed may, therefore, vary along the axial dimension, as is illustrated in FIG. 2.

Disposed on the top of the fiber bed 50 is a carbon dioxide plenum 42 that communicates with the carbon dioxide outlet 17 and with a portion of the fiber bed 50. Disposed on the bottom of the fiber bed 50 is an oxygen plenum 44 that communicates with the oxygen inlet 16 and a portion of the fiber bed 50. The outer periphery 46 of the fiber bed 50, the sidewall 26, and the floor 24 define a bypass channel 52. Although the oxygen inlet 16 and carbon dioxide outlet 17 are depicted in FIG. 1 as being oriented along the vertical axis of the housing 12, other orientations are possible.

The fiber bed 50 can be made out of a variety of materials. In one embodiment of the invention, the fiber bed 50 comprises an annular bundle of gas-permeable, hollow fibers. Both ends of the fiber bundle are potted with a biocompatible adhesive, such as epoxy or polyurethane, and manifolded to enable them to communicate with their respective plenums. In an alternate embodiment of the invention, the fiber bed 50 comprises a disc and a cylindrical bundle assembly of gas-permeable, hollow fibers. The periphery of the disc of the fibers and both ends of the cylindrical fiber bundle are potted. Special manifolds are used to form the gas flow pathway from the cylindrical bundle to the fiber disc. The blood flow enters the fiber bed 50 from the blood inlet 14, travels from one half of the fiber bed 50 to the other half, and exits at the blood outlet 18. Both the blood inlet 14 and the blood outlet 18 can be located at the bottom of the housing 12 in this alternate embodiment. The bypass flow channel 52 remains. In this embodiment, the blood experiences multiple passes through the fiber bed, thereby enhancing the gas exchange rate. In yet another alternative embodiment of the invention, the fiber bed 50 comprises an annular bundle assembly of gas-permeable, hollow fibers of varying inner and/or outer diameter.

The operation of the oxygenator system of FIG. 1 according to an embodiment of the invention will now be described. Oxygen is continuously forced through the oxygen inlet 16, through the oxygen plenum 44, and into the fiber bed 50. When the motor is engaged, the motor rotates the shaft 38, which, in turn, rotates the impeller 36. The rotation of the impeller 36 and its blades 37 creates a flow that draws blood into the blood inlet 14. Blood flow through the system is depicted in FIG. 1 by directional arrows. The flow created by the rotation of the impeller 36 then pushes the blood onto the inner periphery 48 of the fiber bed 50. The blood passes through the fiber bed 50 along a primary flow path depicted by directional arrow 71, during which process the blood receives oxygen that is forced into the fiber bed 50 from the oxygen plenum 44, and the fiber bed 50 removes carbon dioxide from the blood. The removed carbon dioxide diffuses through the fiber bed 50 and into the carbon dioxide plenum 42. Pressure within the carbon dioxide plenum 42 pushes the carbon dioxide out of the housing 12 through the carbon dioxide outlet 17.

The blood exits the fiber bed 50 at its outer periphery 46. The blood can then proceed along one of two paths: (1) the majority of the blood (now oxygenated) leaves the housing 12 through the blood outlet 18, as depicted in FIG. 1 by directional arrows; (2) a portion of the blood flows along the bypass channel 52 to be mixed with incoming blood in a regenerative flow path depicted by directional arrow 72, to be propelled again into the fiber bed 50 by the impeller 36. The volume of blood recirculated through the bypass channel can be increased or decreased by increasing or decreasing the size of the bypass channel 52. As a result of the presence of the bypass channel 52, there are no stagnant flow zones in the space between the housing 12 and the outer periphery 46 of the fiber bed 50, which is a common drawback in prior art pump-oxygenator systems. A further benefit of the bypass channel 52 is to reduce the required surface area of the fiber bundle, hence reducing the overall size of the assembly.

In general, gas is exchanged according to the following convection/diffusion equation:

$$\frac{\partial {C}_{i}}{\partial t} = {D}_{i} {∇}^{2} {C}_{i} - \vec{v} \cdot ∇ {C}_{i} \pm {Σ}_{n} {R}_{i} .$$

In a pumping oxygenator, such as the system 10, the convection-diffusion equation becomes: 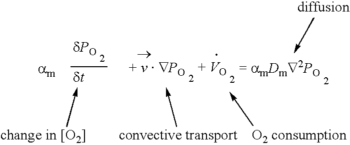
Where αₘ is the oxygen solubility, Dₘ is the oxygen diffusivity, P_{O}_{₂} is the oxygen partial pressure, \dot{V}_{O}_{₂} is oxygen consumption, \vec{ν} is the velocity vector, and t is time. As the second equation shows, convective O₂ transport plays an important role in the total gas exchange to the blood. This is reflected by the convection flux term \vec{ν}·∇P_{O}_{₂}. This is advantageous in reducing the hindrances to the O₂ transfer to blood, considering that the reactions between hemoglobin and O₂ occur fast enough that they may be assumed to reside in equilibrium under most physiological conditions, which means that most transfer resistances come from the gas side. The skilled artisan is aware that gas transfer performance of the instant invention may be evaluated according to standard methods and AAMI standards for blood-pump oxygenators over a predetermined hemodynamic range (i.e., such as for example, up to 6 liter/min of blood).

According to an embodiment of the invention, the blades 37 of the impeller 36 are suitably curved in both the radial and circumferential directions to propel blood both axially and radially, thereby promoting a secondary flow of blood along a toroidal path 70. The conical shape of the hub of the impeller 36 provides uniformity of the radial component of the flow inasmuch as the cross-sectional area between the impeller 36 and the fiber bed 50 diminishes along the axial direction of the flow. The secondary flow encourages the mixing of incoming fresh venous blood and re-circulating oxygenated blood prior to the blood being pumped past the fiber bed 50.

In one implementation of the system depicted in FIG. 1, the nominal flow rate of blood through the system is about 6 liters per minute, the pressure rise from the blood inlet 14 to the blood outlet 18 is about 50-100 torr, and the pressure drop across the fiber bed 50 is about 40 torr.

FIG. 2 illustrates a blood pump-oxygenator in accordance with another embodiment of the present invention. In particular, the system depicted in FIG. 2 is substantially identical or identical in structure and function to the system depicted in FIG. 1, except, for example, that the rotor assembly is located exclusively within the housing 12 in the system of FIG. 2. Moreover, as illustrated in FIG. 2, the system comprises a pair of field coils 60 located within the housing 12, a rotor 62 coupled to the head of the shaft support strut 37, and permanent magnets 64 disposed within the rotor 62. During operation, the coils 60 generate an electromagnetic field that exerts force on the magnets 64, thereby causing the rotor 62 and, consequently, the impeller 36 to rotate. The rotor 62, in this regard, can be affixed to an axi-symmetrical support strut with a ball-and-cup support bearing, while the nose of the rotor 62 is affixed to a very small pivotal bearing. The field coils 60 can be situated to impart also an axial force upon the rotor magnets 64, thereby maintaining contact between the rotor and the support strut. The ball-and-cup support, in this regard, can be made of hard materials with a low coefficient of friction and a high thermal conductivity. The bearing can be washed externally by the free-flowing pumped blood stream to remove the frictional heat generated at the rotary-stationary interface. The high-heat conductivity of the ball-and-socket assembly materials, as well as the relatively small size of the ball-and-cup assembly, allow for an efficient heat transfer between the bearing and the blood stream. In this embodiment, the ceiling 22 does not have an opening for a shaft, as it does not need one. In every other respect, the system depicted in FIG. 2 can operate in the same manner as the system depicted in FIG. 1. In particular, for example, the system can comprise an oxygen inlet and a carbon dioxide outlet (not depicted in FIG. 2).

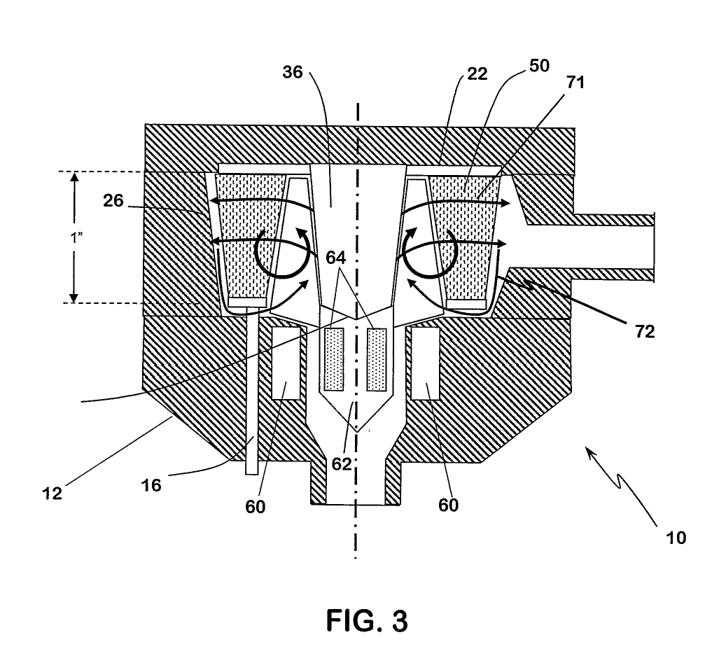

FIG. 3 illustrates a blood pump-oxygenator in accordance with another embodiment of the present invention. In particular, the system depicted in FIG. 3 can be substantially identical or identical in structure and function to the system depicted in FIGS. 1 and 2, except, for example, that the system comprises a fiber bed 50 having a conical profile that assists in equilibrating the cross flow (or radial flow) of blood according to the axial gradient in pressure. Additionally, the system is shown as comprising an oxygen inlet 16. Moreover, the system comprises a sidewall 26, which flairs out into a frustoconical portion to correspond with the shape of the fiber bed 50. During operation, the coils 60 generate an electromagnetic field that exerts force on the magnets 64, thereby causing the rotor 62 and, consequently, the impeller 36 to rotate. The rotor 62, in this regard, can be affixed to an axi-symmetrical support strut with a ball-and-cup support bearing, while the nose of the rotor 62 is affixed to a very small pivotal bearing, as discussed with respect to FIG. 2. The field coils 60 can be situated to impart also an axial force upon the rotor magnets 64, thereby maintaining contact between the rotor and the support strut. Additionally, as discussed with respect to FIG. 2, the ball-and-cup support can be made of hard materials with a low coefficient of friction and a high thermal conductivity and the bearing can be washed externally by the free-flowing pumped blood stream to remove the frictional heat generated at the rotary-stationary interface. The high-heat conductivity of the ball-and-socket assembly materials, as well as the relatively small size of the ball-and-cup assembly, allow for an efficient heat transfer between the bearing and the blood stream. In this embodiment, the ceiling 22 does not have an opening for a shaft, as it does not need one. In every other respect, the system depicted in FIG. 3 can operate in the same manner as the systems depicted in FIGS. 1 and 2.

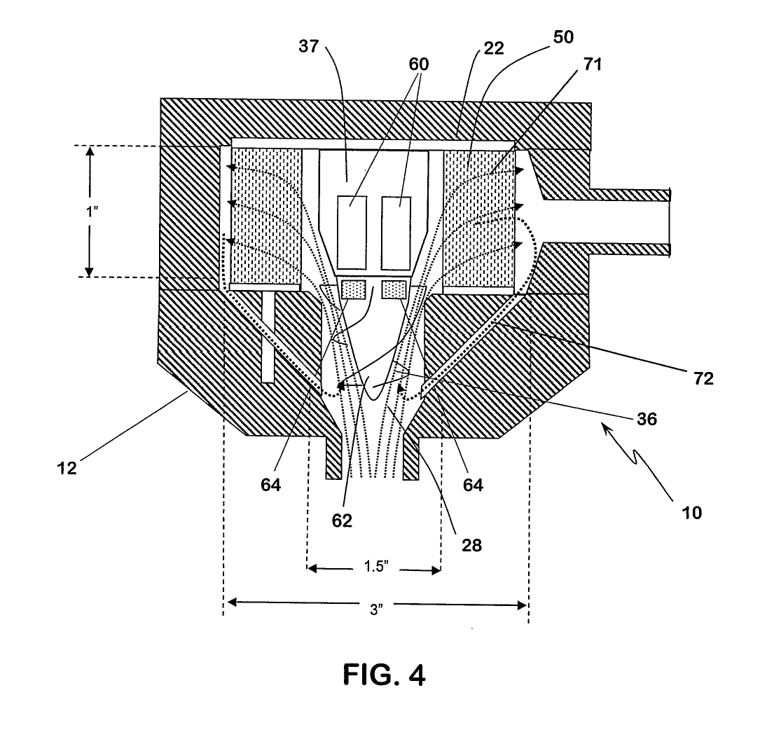

FIG. 4 illustrates a blood pump-oxygenator in accordance with another embodiment of the present invention. In particular, the system depicted in FIG. 4 can be substantially identical or identical in structure and function to the system depicted in FIGS. 1-3, except, for example, that the system comprises a pair of field coils 60, which are located within shaft support strut 37. Additionally, the system comprises bypass channels 52 which direct at least a portion of blood that has passed through fiber beds 50 back to the inlet 28, so that the blood can make another pass through the impeller region and through fiber beds 50. Moreover, impeller 36 is configured as a mixed flow design. During operation, the coils 60 generate an electromagnetic field that exerts force on the magnets 64, thereby causing the rotor 62 and, consequently, the impeller 36 to rotate. The rotor 62, in this regard, can be affixed to an axi-symmetrical support strut with a ball-and-cup support bearing, while the nose of the rotor 62 is affixed to a very small pivotal bearing, as discussed with respect to FIGS. 2 and 3. The field coils 60 can be situated to impart also an axial force upon the rotor magnets 64, thereby maintaining contact between the rotor and the support strut. Additionally, as discussed with respect to FIGS. 2 and 3, the ball-and-cup support can be made of hard materials with a low coefficient of friction and a high thermal conductivity and the bearing can be washed externally by the free-flowing pumped blood stream to remove the frictional heat generated at the rotary-stationary interface. The high-heat conductivity of the ball-and-socket assembly materials, as well as the relatively small size of the ball-and-cup assembly, allow for an efficient heat transfer between the bearing and the blood stream. In this embodiment, the ceiling 22 does not have an opening for a shaft, as it does not need one. In every other respect, the system depicted in FIG. 4 can operate in the same manner as the systems depicted in FIGS. 1-3. In particular, for example, the system can comprise an oxygen inlet and a carbon dioxide outlet (not depicted in FIG. 4).

In one implementation of the present invention, the O₂ transfer is about 250 ml/mn. In another implementation of the present invention, the blood pump-oxygenator has the capacity for paracorporeal implantation. In yet another implementation of the present invention, the blood pump-oxygenator of the present invention is employed for sustained respiratory support for a subject in need thereof.

All references, including publications, patent applications, and patents, cited herein are hereby incorporated by reference to the same extent as if each reference were individually and specifically indicated to be incorporated by reference and were set forth in its entirety herein.

The use of the terms “a,” “an,” “the,” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. It should be understood that the illustrated embodiments are exemplary only, and should not be taken as limiting the scope of the invention.

1. A blood pump-oxygenator system comprising:
a housing;
an impeller disposed within the housing;
a fiber bed disposed between an inner wall of the housing and the impeller; and
a bypass channel that provides a path for blood to be recirculated through the fiber bed.

2. The system of claim 1, wherein the system further comprises at least one blood inlet through which blood can enter the system, and at least one blood outlet through which blood can exit the system.

3. The system of claim 1, wherein the impeller rotates and forces blood that is present in the system to move radially outward through the fiber bed.

4. The system of claim 3, wherein the impeller is operably-connected to a rotor that comprises at least one magnet, and wherein the system further comprises at least one coil that generates an electromagnetic field that exerts force on the at least one magnet and causes the rotor and impeller to rotate.

5. The system of claim 1, wherein the bypass channel is defined by an inner wall of the housing and an outer periphery of the fiber bed.

6. The system of claim 1, wherein the fiber bed oxygenates and removes carbon dioxide from blood that flows through the fiber bed.

7. The system of claim 1, wherein the fiber bed comprises a cylindrical bundle of gas permeable hollow fibers.

8. The system of claim 1, wherein the system further comprises an oxygen inlet channel and an oxygen plenum, which oxygenate blood that passes through the fiber bed.

9. The system of any of claim 1, wherein the system further comprises a carbon dioxide plenum and a carbon dioxide outlet channel, which remove carbon dioxide from blood that passes through the fiber bed.

10. The system of any of claim 1, wherein the system, when operating, is substantially free of stagnant blood flow zones in the space between the housing and the outer periphery of the fiber bed.

11. The system of any of claim 1, wherein the system, when operating, is substantially free of blood boundary layer formation at the fiber bed.

12. A blood pump-oxygenator system comprising:
a housing;
a means for drawing blood into the housing;
a means for removing carbon dioxide from the blood;
a means for adding oxygen to the blood; and
a means for recirculating the blood back through the removing means and the adding means.

13. The system of claim 12, wherein the system further comprises a means for forcing blood drawn into the housing along a toroidal path.

14. The system of claim 12, wherein the system, when operating, is substantially free of stagnant blood flow zones in the space between the housing and the outer periphery of the fiber bed.

15. The system of claim 12, wherein the system, when operating, is substantially free of blood boundary layer formation at the fiber bed.

16. A method for oxygenating blood, which method comprises:
drawing blood into a housing comprising a fiber bed;
forcing the blood to move radially outward through the fiber bed;
adding oxygen to the blood as it moves through the fiber bed; and
for at least a portion of the blood, repeating the drawing, forcing and adding steps.

17. The method of claim 16, wherein the method further comprises removing carbon dioxide from the blood as it moves through the fiber bed.

18. The method of claim 16, wherein the forcing step is performed at least in part by an impeller.

19. The method of claim 16, wherein the fiber bed comprises a cylindrical bundle of gas-permeable hollow fibers.

20. The method of claim 16, wherein the blood moves radially through the fiber bed with substantially no blood boundary layer formation.
